(19) United States
(12) Patent Application Publication
(10) Pub. No.: US 2013/0245469 A1
(43) Pub. Date: Sep. 19, 2013

(54) Pulmonary Arterial Hemodynamic Monitoring for Chronic Obstructive Pulmonary Disease Assessment and Treatment

(71) Applicant: CARDIOMEMS, INC., Atlanta, GA (US)

(72) Inventors: Jay Yadav, Sandy Springs, GA (US); Ralph Jordan, Atlanta, GA (US); Jason White, Smyrna, GA (US)

(73) Assignee: CARDIOMEMS, INC., Atlanta, GA (US)

(21) Appl. No.: 13/843,553

(22) Filed: Mar. 15, 2013

Related U.S. Application Data
(60) Provisional application No. 61/612,758, filed on Mar. 19, 2012.

(51) Int. Cl. A61B 5/0215

(52) U.S. Cl. CPC A61B 5/0215
USPC 600/505; 600/504; 600/300

(57) ABSTRACT

Provided herein are methods for assessing, treating, and for developing new treatments for COPD. Methods can involve obtaining one or more PA hemodynamic readings from a subject with COPD, processing the PA hemodynamic readings to obtain one or more PA hemodynamic parameters, and using the one or more PA hemodynamic parameters to assess, treat, and/or develop new treatments for COPD. The methods can optionally be used to evaluate the progress of (COPD) in a subject, or to predict an outcome in a subject having COPD.

### CROSS-REFERENCE TO RELATED APPLICATIONS

This application claims the benefit of U.S. Provisional Patent Application No. 61/612,758, filed on Mar. 19, 2012, entitled “Pulmonary Arterial Pressure for Chronic Obstructive Pulmonary Disease Assessment and Treatment,” the disclosure of which is expressly incorporated herein by reference in its entirety.

### BACKGROUND

Chronic obstructive pulmonary disease (COPD) is a common lung disease. It is a long term disease that makes is difficult for afflicted individuals to breathe. There is no cure for COPD. New and better methods for assessing COPD in subjects, for treating subjects with COPD, and for developing new treatments for COPD are needed. These efforts are hindered by a low probably of therapeutics reaching the market, a long development duration, a rudimentary understanding of the biological genesis of COPD, in adequate in vivo models, poorly validated biomarkers, and inefficient physiological and clinical endpoints.

The heart and lungs function together in an intimately coupled loop, so it is important to understand the effects of COPD on the heart. The primary effect of COPD on heart function is by through hypoxic pulmonary vasoconstriction. COPD causes hypoxia at the alveolar—pulmonary arterial capillary interface, which causes hypoxic pulmonary vasoconstriction. Hypoxic pulmonary vasoconstriction causes elevated resistance to blood flow through the pulmonary arterial capillaries, which elevates pulmonary arterial pressure, and increases the right ventricular (RV) workload and energy expended per cardiac cycle. An elevated RV workload results in a reduced maximum cardiac output and reduced exercise capacity. Over time, elevated right ventricular workload progressively results in decreasing maximum cardiac output, acute episodes of fatigue or weakness, right ventricular failure (cor pulmonale), cardiac insufficiency at rest, and death.

The effects of COPD on pulmonary hemodynamics are most broadly evident under conditions that may cause an oxygen deficit, such as during exertion or with sleep disorder breathing. In advanced COPD, abnormal pulmonary hemodynamics may also be observed at rest.

Primary clinical metrics for COPD do not focus on the cardiac effects of the disease. Common lung function tests include spirometry, diffusion tests, lung imaging tests, and body plethysmography. These tests do not provide information specific to cardiac effects of the disease. While exercise capacity tests, such as 6MW and VO2 max, can measure exercise tolerance to evaluate the global effect of the disease on the body, which can includes its effect on cardiac function, these tests lack specificity about cardiac function because of numerous other factors which can influence the patient's response.

### SUMMARY

Provided herein are methods for assessing, treating, and for developing new treatments for COPD.

For example, provided herein are methods of predicting the progression or improvement of chronic obstructive pulmonary disease (COPD) in a subject, or an outcome in a subject having COPD. The methods can comprise obtaining one or more pulmonary arterial (PA) hemodynamic readings from the subject. The PA hemodynamic readings can be collected under any of a wide range of physical activity state conditions, including, for example, rest, sleep, activities of daily living, or exercise. The PA hemodynamic readings can comprise a high-fidelity, real-time PA hemodynamic waveform. Pulmonary arterial hemodynamic parameters, including systolic PA pressure, diastolic PA pressure, mean PA pressure, heart rate, respiratory rate, cardiac output (CO), total pulmonary resistance, and stroke volume, can be determined from information contained within the PA hemodynamic waveform, as described previously in the art. A change (e.g., increase or decrease) in one or more PA hemodynamic parameters as compared to a standard can indicate, for example, disease progression, disease improvement, or clinical outcome.

Also provided are methods of predicting the outcome or for evaluating progress or improvement in a subject having COPD. The methods comprise administering an agent to the subject, obtaining one or more PA hemodynamic readings from the subject, and comparing the one or more PA hemodynamic readings obtained to a standard to indicate the progression of COPD, indicate the improvement of COPD, or to predict an outcome in a subject having COPD.

Further provided are methods for determining an effective dose of an agent for the treatment of COPD. The methods comprise administering the agent to a subject having COPD, obtaining one or more PA hemodynamic readings from the subject, and comparing the one or more PA hemodynamic readings obtained to a standard. A lowered obtained reading compared to the standard indicates the effective dosage of the agent for treatment of COPD.

Also provide are methods for treating a subject with COPD. The methods comprise administering a pharmaceutical agent to the subject, obtaining one or more PA hemodynamic readings from the subject, and comparing the one or more PA hemodynamic readings obtained to a standard. A lowered reading compared to the standard indicates treatment of COPD.

Also provided are methods for optimizing an effective dose and or dosing regimen of an agent for the treatment of COPD in a subject. The methods comprise administering a predetermined dosage of the agent to the subject by a predetermined dosing regimen, obtaining one or more PA hemodynamic readings from the subject, comparing the one or more PA hemodynamic readings obtained to a standard to determine the effect of the agent on PA hemodynamics in the subject, and adjusting the dosage or dosing regimen to obtain the optimized effective dose or dosing regimen.

Also provided are ambulatory systems for the collection of hemodynamic readings. The ambulatory systems can be used in conjunction with the methods described herein. In certain embodiments, the ambulatory systems are used to obtain ambulatory PA hemodynamic readings from a patient (e.g., during exercise).

The details of one or more embodiments are set forth in the accompanying drawings and the description below. Other features, objects, and advantages will be apparent from the description and drawings, and from the claims.

### DESCRIPTION OF DRAWINGS

FIG. 1 is a plot of cardiac output in both the resting state (solid line) and upon exercise (broken dash line) as a function of the clinical progression of heart failure, which may occur as a result of COPD induced pulmonary hypertension.

FIG. 2 shows comparison between the PA hemodynamic waveform obtained using right heart catheterization (top) and CARDIOMEMS implantable pressure sensor (bottom). The waveform obtained using RHC shows some undesired whip or overshoot that is believed to exceed actual systolic and diastolic pressures and which can lead to error and uncertainty. The CARDIOMEMS sensor waveform on the bottom, in contrast, exhibits high-fidelity through its smooth and undistorted waveform.

FIG. 3 is a block diagram of an embodiment of a ambulatory system which can be used in conjunction with the methods described herein.

FIG. 4 is a graph showing mortality rates stratified by mean PAP. This graph shows elevated mortality rates for COPD patients with elevated mean PAP.

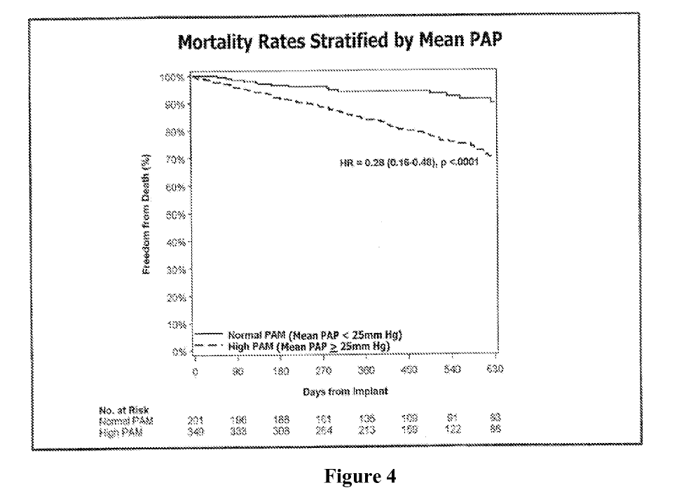

FIG. 5 is a graph showing rates of hospitalization by baseline PA mean pressures. This graph shows elevated hospitalization rates with elevated mean PAP.

FIG. 6 is a graph showing disproportionate increases in PAP during exercise for COPD patients. This graph demonstrates how COPD patients with normal resting PAP may have elevated PAP during and exertion and normal activities of daily living. The integrated/average PAP experienced by the heart over time is an indicator of the ventricular workload.

### DETAILED DESCRIPTION

Provided herein are methods for assessing, treating, and for developing new treatments for COPD. Methods can involve obtaining one or more PA hemodynamic readings from a subject with COPD, processing the PA hemodynamic readings to obtain one or more PA hemodynamic parameters, and using the one or more PA hemodynamic parameters to assess, treat, and/or develop new treatments for COPD. The methods can optionally be used to evaluate the progress of (COPD) in a subject, or to predict an outcome in a subject having COPD.

The methods described herein are optionally used to develop a therapeutic agent for the treatment of COPD. The term “developing” or “development” as used herein in reference to therapeutics are broad terms that include, by way of example and not limitation, prospective design, or selection, of one or more potential therapeutic methods or compounds for COPD, or retrospective study of one or more therapeutic methods or compounds or design of studies of such therapeutic methods or compounds. In this regard, to develop or development of a therapeutic can include, for example, changes to an active ingredient or formulation and also includes, for example, study design for a therapeutic. Optionally, study design is for a clinical trial and development of a study design can include establishing trial metrics or trial durations.

The methods described herein can also be used to identify a candidate therapeutic agent for administration to a patient for the treatment of COPD. In some embodiments, one or more of the PA hemodynamic parameters can be correlated with the candidate therapeutic agent for the treatment of COPD to indicate a predicted change in one or more PA hemodynamic parameters in the patient that would result from administration of the candidate agent. The predicted change can be used to indicate the predicted effect of the candidate agent on one or more PA hemodynamic parameters of the subject.

A change in one or more PA hemodynamic parameters, (e.g., PAP values) resulting from the administration of the therapeutic agent can be identified, the change indicating an effect of the therapeutic agent on the PA hemodynamic parameter of the subject. Optionally, the PA hemodynamic data comprises at least one PA hemodynamic parameter measured in a subject prior to administration of the therapeutic agent. Optionally, the PA hemodynamic data comprises at least one PA hemodynamic parameter measured in a subject concurrent with administration of the therapeutic agent. Optionally, the PA hemodynamic data comprises at least one PA hemodynamic parameter measured in a subject subsequent to administration of the therapeutic agent. Optionally, the PA hemodynamic data comprises at least one PA hemodynamic parameter measured in a subject prior to administration of the therapeutic agent and at least one PA hemodynamic parameter measured in a subject subsequent to administration of the therapeutic agent. In some aspects, one or more additional therapeutic agents are administered to the subject prior to, concurrently with, or subsequent to the therapeutic agent.

Optionally, the therapeutic agent can be modified to increase the indicated effect. For example, if the indicated effect is desired (e.g., therapeutic efficacy for COPD is observed), the structure of the therapeutic agent can be modified to increase the indicated effect. The therapeutic agent can also be modified to decrease the indicated effect. For example, if the indicated effect is not desirable, then the structure of the therapeutic agent can be modified to decrease the indicated effect. Moreover, an administration characteristic of the therapeutic agent can be modified to increase or decrease the indicated effect.

The administration characteristic can be selected from the group consisting of dosage amount, number of doses, timing of doses, route of administration, and total dosage. When the indicated effect is to be increased or decreased, one or more portions of the therapeutic agent responsible for the indicated effect can be determined. Optionally, a second therapeutic agent including the one or more portions of the therapeutic agent responsible for the indicated effect can be designed.

The indicated effect can used to assess safety of the therapeutic agent for administration to a mammal or population thereof for the treatment of COPD. In some examples, the indicated effect is used to assess the toxicity of the therapeutic agent for administration to a mammal or population thereof. Optionally, the toxicity can be cardiac toxicity. The indicated effect can also be used to assess the efficacy of the therapeutic agent for administration to a mammal or population thereof to treat COPD. The indicated effect can also be used to predict the effect or effects of the therapeutic agent or agents having the same or similar pharmacological characteristics on the PA hemodynamic parameter, on COPD, or combinations thereof. Optionally, the indicated effect is used to predict the effect or effects of the therapeutic agent or agents having the same or similar pharmacological characteristics on a PA hemodynamic parameter of a mammal. In some aspects, the indicated effect is used to determine an end point for a clinical trial of a therapeutic agent for COPD.

The described methods can further comprise determining one or more characteristics of the subject. The determined characteristic can be a physical, physiologic, metabolic, chronological, disease state, drug administration history, medical history, or genetic characteristic. The characteristic can be correlated with the indicated effect in the subject. The correlation of the characteristic and the indicated effect in the subject can be used to select one or more additional subjects for administration of the therapeutic agent or for a therapeutic agent having the same or similar indicated effect. The correlation of the characteristic and the indicated effect can also be used to select one or more additional subjects to participate in a clinical trial for the therapeutic agent or for a therapeutic agent having the same or similar indicated effect.

These determinations can be used to facilitate development of a therapeutic by efficiently identifying preferred dosages that are correlated to improved physiological data for clinical trials. The determinations can also be used for determining proper dosages of commercial products for general and specific populations of subjects. For example, the described methods can be used to arrive at dosing levels based on patient/subject profiles including, but not limited to, the response of one or more PA hemodynamic parameters (e.g., PAP) to a study or commercial drug or with other hemodynamic metrics alone or in combination with characteristics such as age, weight, disease state (e.g., severity of COPD), and concurrent drug administration or drug-drug interaction. Example methods for facilitating development of therapeutics include methods for evaluating progress or outcome in COPD subjects, and for evaluating treatment in COPD subjects. These methods can also be used in clinical treatment and management of subjects.

As described above, methods can involve comparing one or more PA hemodynamic parameters to a standard value. A standard can, for example, be a previous PA hemodynamic value from the same subject, a different subject, or a value based on PA hemodynamic values measured from a population of subjects. Optionally, the standard is based on a plurality of PA hemodynamic readings previously taken from the same subject.

Example methods can include obtaining one or more PA hemodynamic readings from a subject with COPD, processing the PA hemodynamic readings to obtain one or more PA hemodynamic parameters, and using the one or more PA hemodynamic parameters to assess, treat, and/or develop new treatments for COPD. The PA hemodynamic parameter can include systolic PA pressure, diastolic PA pressure, mean PA pressure, heart rate, respiratory rate, cardiac output (CO), total pulmonary resistance, stroke volume, or combinations thereof. The PA hemodynamic parameters can be compared to corresponding standard PA hemodynamic parameters. The comparisons of PA hemodynamic parameters can be used to monitor COPD progression in the subject and/or predict the progression or improvement of COPD in the subject, and/or predict a clinical outcome in a subject having COPD

For example, a lowering trend in PAP values over time in, optionally, a subject having COPD can indicate improvement in the COPD condition. This can also optionally indicate improved outcome in the subject, such as, for example, reduced hospitalizations, lower mortality, or lower morbidity. Conversely, an increased PAP reading as compared to the standard can indicate progression of the COPD in the subject or predict a worse outcome for the subject having COPD (e.g., reduced hospitalizations, lower mortality, or lower morbidity). An increased maximum cardiac output reading as compared to the standard can indicate improvement of the COPD in the subject and/or can indicate an improved outcome for the subject having COPD. A decreased maximum cardiac output reading as compared to the standard can indicate progression of the COPD in the subject and/or can indicate a worse outcome for the subject having COPD.

As discussed above, the predicted outcome for the subject with COPD can, for example, be mortality rate or hospitalization rate. Optionally, the predicted outcome is mortality rate, wherein the mortality rate is either reduced or increased for the subject. By way of an example, if PAP is increased compared to a standard, the mortality rate is expected to be increased. By way of another example, if the PAP is decreased compared to a standard, the mortality rate is decreased. Optionally, the predicated outcome is hospitalization rate, wherein the hospitalization rate is either reduced or increased for the subject. By way of an example, if PAP is increased compared to a standard, the hospitalization rate for the subject is expected to be increased. By way of another example, if PAP is decreased as compared to a control, the hospitalization rate for the subject is expected to be decreased. A reduced expected hospitalization rate can result from, for example, a reduction in hospitalizations for heart failure or respiratory events.

Method for treating subjects with COPD and/or evaluating the treatment of a subject having COPD can further include treating the subject for COPD. Treatment of subjects with COPD can include the administration of therapeutic agents (e.g., drugs) to the subject and/or surgical intervention.

In some embodiments, COPD may be treated with a bronchodilator. Bronchodilators are medicines that relax smooth muscle around the airways, increasing the caliber of the airways and improving air flow. They can reduce the symptoms of shortness of breath, wheeze, and exercise limitation, resulting in an improved quality of life for people with COPD. Bronchodilators are usually administered with an inhaler or via a nebulizer. There are two major types of bronchodilators: β2 agonists and anticholinergics. Each type may be either long-acting (with an effect lasting β2 hours or more) or short-acting (with a rapid onset of effect that does not last as long).

β2 agonists stimulate β2 receptors on airway smooth muscles, causing them to relax. There are several β2 agonists available. Albuterol and terbutaline are widely used short acting β2 agonists and provide rapid relief of COPD symptoms. For example, the inhaled form of terbutaline starts working within 15 minutes and can last up to 6 hours. Salbutamol sulfate is usually given by the inhaled route for direct effect on bronchial smooth muscle through a metered dose inhaler (MDI), nebulizer, or other proprietary delivery devices. In these forms of delivery, the maximal effect of salbutamol can take place within five to 20 minutes of dosing. It can also be given intravenously. Long acting β2 agonists (LABAs) such as salmeterol and formoterol (eformoterol) may be used as maintenance therapy and lead to improved airflow, exercise capacity, and quality of life. For example, salmeterol is available as a dry powder inhaler that releases a powdered form of the drug. Formoterol is currently marketed as a dry-powder inhaler (DPI), a metered-dose inhaler (MDI), an oral tablet, and an inhalation solution, under various trade names including FORADIL/FORADILE (Schering-Plough, Novartis), OXEZE/OXIS (AstraZeneca), ATOCK (Astellas), ATIMOS (Modulite, Chiesi), and PERFOROMIST (Dey). In some cases, the β2 agonists may be combined with a steroid. For example, the combination of fluticasone and salmeterol are marketed as ADVAIR (GlaxoSmithKline).

Anticholinergic drugs cause airway smooth muscles to relax by blocking stimulation from cholinergic nerves. For example, ipratropium bromide is supplied in a canister for use in an inhaler or in single dose vials for use in a nebulizer to provide short-acting rapid relief of COPD symptoms. Tiotropium is a long-acting (24 hour) anticholinergic whose regular use is associated with improvements in airflow, exercise capacity, and quality of life.

In addition to the above, corticosteroids may be used in tablet or inhaled form to treat and prevent acute exacerbations of COPD. Well-inhaled corticosteroids (ICS) have been shown to decrease acute exacerbations in those with either moderate or severe COPD. Antibiotics, in particular macrolides, such as azithromycin, may also be used reduce the number of exacerbations in those who have two or more a year.

Supplemental oxygen or oxygen therapy can improve oxygen saturation levels, allowing patients with COPD or low oxygen levels to maintain their mobility and increase their ability to complete activities of daily living (ADL), such as exercise, household chores, shopping, etc. Long-term oxygen therapy for at least 16 hours a day can improve the quality of life and survival for people with COPD and arterial hypoxemia or with complications of hypoxemia such as pulmonary hypertension, cor pulmonale, or secondary erythrocytosis. High concentrations of supplemental oxygen can lead to the accumulation of carbon dioxide and respiratory acidosis for some people with severe COPD; lower oxygen flow rates are generally safer for these individuals. Another safety issue concerning the use of oxygen for patients with COPD is smoking, because oxygen can act as an oxidizing agent.

COPD can also be treated using pulmonary rehabilitation, which is a coordinated program of exercise and disease management. Pulmonary rehabilitation has been shown to improve shortness of breath and exercise capacity. It has also been shown to improve the sense of control a patient has over their disease as well as their emotions.

Surgery is sometimes helpful for COPD in selected cases. A bullectomy is the surgical removal of a bulla, a large air-filled space that can squash the surrounding, more normal lung. Lung volume reduction surgery involves removal of parts of the lung that are particularly damaged by emphysema, allowing the remaining, relatively good lung to expand and work better. Lung transplantation is also sometimes performed for severe COPD, particularly in younger individuals.

Examples of treatments that can be monitored and/or studies using the methods described herein include the effects of a fixed dose combination of Tiotropium and Olodaterol on exercise endurance time during constant work load cycle testing in COPD and the use of ergonomic functioning to ensure/support superior efficacy of combination therapy. Another example is optionally, Tiotropium in early COPD disease, for example to support proposition that tiotropium decreases lung function decline and reverses disease progression in early stage COPD. In another example, an inhaled corticosteroid can be withdrawn during treatment of COPD to support that there is no need for continued corticosteroid treatment in combination with a tiotropium background therapy. Another example, use of tiotropium and olodaterol to demonstrate the efficacy in terms of exertional PAP to offset first to market impact of LABA/LAMA(s). Another example, to assess Micardis PAP reduction profile. The methods described herein can also be used to facilitate the selection and/or validation and/or use of biomarkers associate with COPD.

Methods of treatment can involve treating a subject with COPD (e.g., by administering an agent to the subject), obtaining PA hemodynamic readings from the subject, processing the PA hemodynamic readings to obtain one or more PA hemodynamic parameters, and using the one or more PA hemodynamic parameters to assess, treat, and/or develop new treatments for COPD. For example, the obtained PA hemodynamic parameters subsequent to the administration of the treatment can, for example, be compared to standard values for the corresponding hemodymanic parameters prior to (or earlier in the course of) administration of the treatment) to assess treatment efficacy.

The treatment (e.g., the pharmaceutical agent), for example, the agent administered, dosage, timing, protocol, or the like, can be modified or maintained after determining the effect of the treatment of the COPD in the subject. For example, administration of the pharmaceutical agent is optionally maintained at its current dosage and or dosing regimen when improvement is indicated. In another example, the pharmaceutical agent is changed, or the dosage or dosing regimen modified when improvement is not indicated. In another example, the information obtained on PAP, CO, Total Pulmonary Resistance (TPR=mPAP/CO) or any combination of these parameters are used to determine an effective dosage or administration protocol for the agent or to evaluate the same, for example, in a clinical trial setting. In the case of COPD, therapeutic goals optionally include lowering or maintaining PAP, lowering or maintaining TPR, and/or increase or maintain CO.

For example, in the case of COPD, a decreased PAP reading as compared to the standard following administration of a treatment can indicate improvement of the COPD in the subject and/or can indicate an improved outcome for the subject having COPD. Similarly, an increased maximum cardiac output reading as compared to the standard following administration of the treatment can indicate improvement of the COPD in the subject and/or can indicate an improved outcome for the subject having COPD. In these cases, methods can further include continued administration of the treatment to the subject. Similarly, an increased PAP reading and/or a decreased maximum cardiac output reading as compared to the standard following administration of a treatment can indicate no beneficial effect of the treatment on COPD in the subject.

Optionally, the methods can further comprise selecting a subject with COPD or a subject at risk for developing COPD. The subject at risk for developing COPD can be a tobacco smoker, a subject with a genetic susceptibility to developing COPD (e.g., a subject with a family history of COPD or a subject with a genetic mutation, such as a alpha 1-antitrypsin deficiency, which places the subject at increased risk for the development of COPD), or a subject with an occupational history which includes prolonged exposure to dusts (e.g., a coal miner, a gold miner, or a textile worker) or chemicals such as cadmium, isocyanates, and fumes from welding).

Obtaining PA Hemodymanic Parameters

PA hemodynamic parameters can be determined by analysis of the PA pressure waveform to determine the average maximum waveform values for systolic PAP, average minimum waveform values for diastolic PAP, and average of all waveform values for mean PAP.

Heart Rate is optionally determined in the following manner: The timing of repetitive relevant hemodynamic events within the cardiac cycle, such as optionally the average time interval between consecutive systolic or diastolic pressure values, is used to determine the average time interval between beats. The average time interval between beats is optionally used to determine the average heart rate as 60 (s/min.)/average time interval between beats (s/beat) to determine the average heart rate in beats per minute.

Cardiac Output (CO) is estimated based on the pulmonary arterial pressure waveform using the following approach. For each cardiac cycle, the pulmonary arterial pressure waveform P(t), comprised of an array of consecutive discrete paired pressure (p) and time (t) values, P(t)={(p₁,t₁), (p₂,t2) . . . }, is optionally analyzed to identify the following relevant reference pressure and time points for each beat:
P1,T1 pressure and time at start of systole and end of diastole.
P2,T2 pressure and time at end of the RV incident wave/beginning of reflected wave indicated as first upslope pressure incisura after the systolic upslope dP/dT max or alternatively at the maximum of the pressure beat.
P3,T3 pressure and time at end of systole/end of outflow demarcated by the dicrotic notch/pulmonic valve closure.

Optionally the pressure waveform features associated with the RV incident pressure wave, and time parameters during systole are used to determine a proportional estimation of stroke volume.

The proportional estimate of stroke volume for each beat, can be determined in a variety of ways, including but not limited to the following:
a. P2−P1
b. √P2
c. √(P2−P1)
d. √(average(P2,P3))
e. √P2×(T3−T1)
f. √(P2−P1)×(T3−T1)
g. √(average(P2,P3))×(T3−T1)
h. √P2×(T3−T2)
i. √(P2−P1)×(T3−T2)
j. √(average(P2,P3))×(T3−T2)
k. dP/dT max value between T1 and T2
l. Integration of P(t)−P1, from T1 to T2.
m. Integration of the first derivative of the pressure waveform, dP(t)/dT, from T1 to T2.
n. Integration of √(P(t)−P1), from T1 to T2.
o. Integration of √dP(t)/dT, from T1 to T2.
   Alternatively, for estimates which integrate pressure changes over time and which have a downstream reference pressure (P1) (l and n) may be replaced in the equation with a value that gradually increases as the pulmonary vasculature volume increases during filling along the line that is defined by endpoints P1,T1 and P3,T3, according to the equation;

P(t)_{[P1,T1 to P3,T3]}=P1+[(P3−P1)/(T3−T1)]×(t−T1)

Accordingly, the proportional estimate of stroke volume can also be performed as follows:
p. Integration of P(t)−P(t)_{[P1,T1 to P3,T3]} from T1 to T2.
q. Integration of √(P(t)−P(t)_{[P1,T1 to P3,T3]}), from T1 to T2.
   Alternatively, for estimates that integrate pressure changes or the square root of pressure changes over time (l, n, p, and q), integration over the timeframe from T2 to T3 may also be included, with exclusion of the portion of the waveform which is attributed to the reflected wave. The reflected portion of the waveform is delineated by the line defined by endpoints P2,T2 and P3,T3, defined by the equation;

P(t)_{[P2,T2 to P3,T3]}=P2+[(P3−P2)/(T3−T2)]×(t−T2)

Accordingly, the proportional estimate of stroke volume can also be performed as follows:
r. Integration of P(t)−P1, from T1 to T2 plus integration of P(t)_{[P2,T2 to P3,T3]}−P1 from T2 to T3.
s. Integration of √(P(t)−P1), from T1 to T2 plus integration of √(P(t)_{[P2,T2 to P3,T3]}−P1) from T2 to T3.
t. Integration of P(t)−P(t)_{[P1,T1 to P3,T3]} from T1 to T2 plus integration of P(t)_{[P2,T2 to P3,T3]}−P(t)_{[P1,T1 to P3,T3]} from T2 to T3.
u. Integration of √(P(t)−P(t)_{[P1,T1 to P3,T3]}), from T1 to T2 plus integration of √(P(t)_{[P2,T2 to P3,T3]}−P(t)_{[P1,T1 to P3,T3]}) from T2 to T3.

A proportional estimate of stroke volume (SV_{prop.est}). is determined as the average of the compiled proportional estimates from at least one cardiac cycle. The duration or number of cardiac cycles for the averaging window can be configured as appropriate to the application. The minimum average length preferably exceeds the length of at least one respiratory cycle in order to average variation attributable to the respiratory cycle.

A proportional estimate of cardiac output is determined by multiplying the average proportional estimate of stroke volume by the measured average heart rate.

An initial reference cardiac output measurement is performed for method calibration, typically during the sensor implantation procedure using a clinically accepted measurement method such as thermodilution, modified Fick, or Fick. During the same measurement session, one or more wireless pulmonary arterial pressure waveform readings are collected. An initial patient specific constant term (Aᵢ) is calculated as the ratio of the reference cardiac output measurement and the proportional estimate of cardiac output determined during the same measurement session. This initial patient specific constant term indirectly determines the effects of other relevant parameters such as the RV outflow tract cross sectional area (CSA) and the initial characteristic impedance during the calibration measurements (Z_{o,i}); =CSA×Z_{o,i}. This initial patient specific constant term is used as a calibration factor that is multiplied by the proportional estimate of cardiac output to produce the initial estimated cardiac output value; COₑₛₜ_{_{—}}_{Zo,i}.=Aₒ×SV_{prop.est}×HR.

As a corollary to Ohm's law for electrical circuits, the relationship between pressure changes (ΔP) and flow (Q) is governed by flow impedance (Z); ΔP(t)=Z(t)×Q(t). Thus, the pressure based proportional estimate of stroke volume and cardiac output is valid only without relevant changes in impedance. This assumption is valid during short term assessments, as would be the case during an initial calibration reading of, for example, 18s length. However, flow impedance may change over longer timeframes between readings. For this reason, also accounting for changes in impedance between readings is expected to yield improved model performance.

Flow impedance has frequency and non-frequency dependent components.

The frequency dependent components of impedance are primarily associated with reflected, propagated pressure waves which dynamically influence the relationship between pressure and flow. These frequency dependent effects are incorporated into the model through the previously presented use of the first upslope incisura as the T2 timepoint in the initial stroke volume estimate. For example, more prominent reflected pressure waves or faster pressure wave propagation associated with increased pulmonary vascular impedance results in an earlier first upslope incisura, which reduces the proportional estimate of stroke volume. Conversely, less prominent reflected pressure waves or slower pressure wave propagation associated with reduced pulmonary vascular impedance results in a later first upslope incisura, which results in an increased proportional estimate of stroke volume.

In one example, changes in the non-frequency dependent, characteristicimpedance after initial calibration are incorporated in order to achieve improved accuracy in the stroke volume and cardiac output estimates without need for recalibration over extended timeframes.

The physiological basis for the non-frequency dependent impedance change estimate, hereafter described as impedance change, ΔZ(t), is presented as follows. The input stroke volume is a bolus injection that is introduced into the pulmonary vasculature during a minor portion of the total cardiac cycle, indicated by the timeframe from start of systole to the first upslope incisura (T2−T1). Conversely, the outgoing stroke volume leaves the pulmonary vascular through the pulmonary capillaries approximately continuously throughout the entire cardiac cycle. During the relatively short input stroke volume timeframe, a minor portion of the pulmonary vascular blood volume leaves the pulmonary arterial vasculature through the pulmonary capillary beds, approximately (T2−T1)/(Time interval between beats), approximately <10% of the total outgoing stroke volume. Accordingly, changes in resistance have only a minor impact on the characteristic impedance during the short T2-T1 timeframe, and the pulmonary vasculature during the T2-T1 timeframe can be fairly approximated as a closed system, with the major component of characteristic impedance determined by the pulmonary vascular compliance, ΔV/ΔP.

With increased mean, systolic, or diastolic average pulmonary arterial pressures, the pulmonary arterial wall strain modulus increases, and compliance decreases in a predictable manner, due to the mechanical properties of the pulmonary vessels. (Pasierski T J, CHEST 1993). Based on this relationship, changes in mean, systolic, or diastolic average pulmonary pressures from baseline to a follow-up measurement can be used to infer proportional changes in impedance from baseline values.

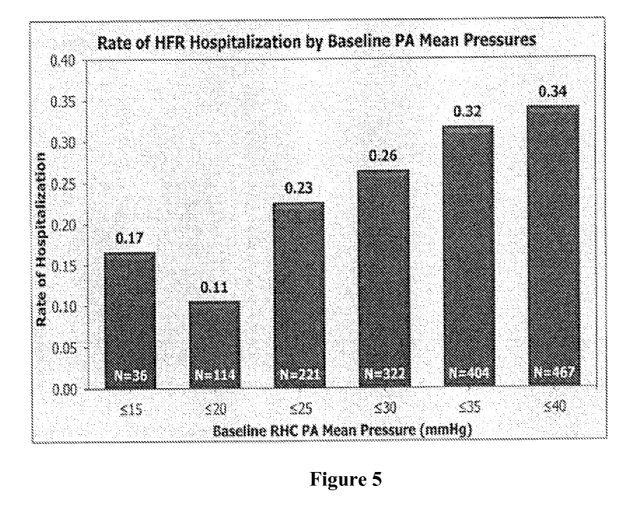

The relationship between average pulmonary arterial pressure changes and proportional impedance changes was determined heuristically using data obtained from 54 NYHA Class III Heart Failure patients with 116 follow-up sets of reference cardiac output measurements and wireless pulmonary arterial pressure measurements performed a mean±st. dev. (min., max) of 463±422 (48, 1281) days post implant. The estimated Cardiac Output for follow-up measurements was determined using the initially determined constant term from baseline (Aᵢ), which does not account for changing impedance: CO_{est-Zo,i}=Aₒ×SV_{prop.est}×HR. The effect of changing impedance was inferred using a scatter plot of the proportional residual error at follow-up, defined as (COₑₛₜ_{_{—}}_{Zo,i}−CO_{ref})/COₑₛₜ_{_{—}}_{Zo,i}, vs. changes in average pulmonary arterial pressure from baseline. There was an observed trend of residual error with respect to mean pressure changes for mean pressure changes in the provided example. The pattern of residual error as a function of mean pulmonary arterial pressure change over the threshold value from baseline to follow-up, ΔmPAP, was characterized using an exponential regression, with constant term coefficient, B=−0.025, by the following equation (FIG. 5):

Curve fit for proportional residual error at follow-up=(COₑₛₜ_{_{—}}_{Zo}−CO_{ref})/COₑₛₜ_{_{—}}_{Zo}=1−e^{(B×ΔmPAP)}1−e^{(-0.025×ΔmPAP)}

Predicted residual error=COₑₛₜ_{_{—}}_{Zo}−CO_{ref}=COₑₛₜ_{_{—}}_{Zo}×(1−e^{(-0.025×ΔmPAP)})

In order to incorporate the impedance change effect, the predicted residual error is subtracted from COₑₛₜ_{_{—}}_{Zo,i} to produce a CO estimate with impedance change effects incorporated, COₑₛₜ_{_{—}}_{Z(t).}

COₑₛₜ_{_{—}}_{Z(t)=CO}ₑₛₜ_{_{—}}_{Zo,i}×(1−e^{(-0.025×(ΔmPAP)})=COₑₛₜ_{_{—}}_{Zo,i}×e^{(-0.025×ΔmPAP)}

Finally, it should be noted that model coefficients, such as but not limited to the mean pressure threshold value and exponential curve fit coefficient (B), can be used to optimize results based on best fit for a given population or individual patient, using sets of pressure waveform data and CO reference values measured at more than one time with relevant CO change between measurements.

Exercise

The primary effect of COPD on heart function is by through hypoxic pulmonary vasoconstriction. COPD causes hypoxia at the alveolar—pulmonary arterial capillary interface, which causes hypoxic pulmonary vasoconstriction. Hypoxic pulmonary vasoconstriction causes elevated resistance to blood flow through the pulmonary arterial capillaries, which elevates pulmonary arterial pressure, and increases the right ventricular (RV) workload and energy expended per cardiac cycle. An elevated RV workload results a reduced maximum cardiac output and reduced exercise capacity. Over time, elevated right ventricular workload progressively results in decreasing maximum cardiac output, acute episodes of fatigue or weakness, right ventricular failure (cor pulmonale), cardiac insufficiency at rest, and death.

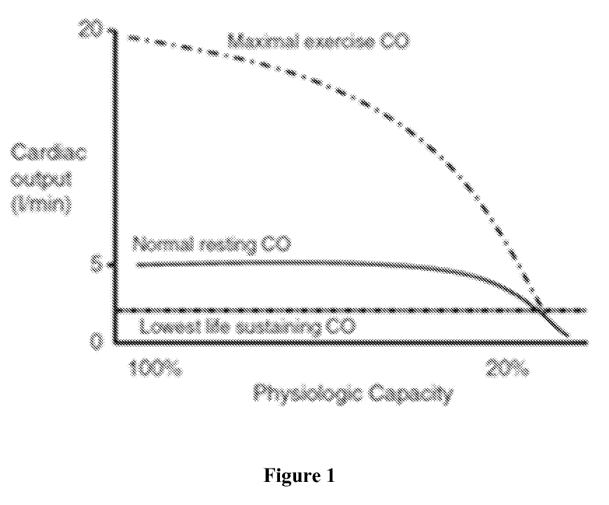

FIG. 1 illustrates cardiac output in both the resting state (solid line) and with maximal exercise (broken dash line) relative to the heart function across the full range of heart failure. In the context of COPD, right ventricular failure may occur as an effect of pulmonary hypertension induced by COPD. This severity of this effect may be effectively tracked effectively by changes in the maximal cardiac output, while there may be no change in resting cardiac output.

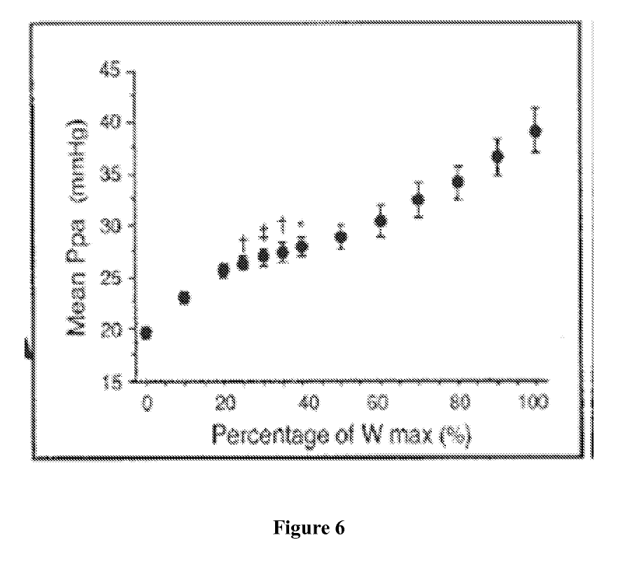

Disproportionate increases in PAP may also be observed during exercise for COPD patients, as shown in FIG. 6, even if resting PAP pressures are normal. In a similar manner to use of exercise CO, abnormal increases in PAP during exercise can be used to assess the severity of COPD on heart function more effectively, and at an earlier stage in the course of the disease progression than would otherwise be possible with PAP measurements at rest.

In COPD, elevated resistance to flow through the lungs is the cause of elevated PAP, elevated RV workload, and reduced maximum CO. Total pulmonary resistance is also determined as the ratio of mPAP/CO, and is expected to be another highly sensitive metric which takes into account changes in both mPAP and CO. Thus, the progression of COPD can be more evident when analyzing PA hemodynamic parameters, such as CO, PAP, and TPR, measured from a subject during exercise.

Accordingly, in the case of the methods described herein, the PA hemodynamic readings can be obtained from the subject having COPD during exercise (e.g., physical exertion such as walking on a treadmill or riding an exercise bike). Methods can further include instructing a subject having COPD to exercise prior to and/or during the measurement of PA hemodynamic readings.

While described with respect to COPD above, the severity of heart failure (HF) generally, and the severity of right heart failure in particular, can be effectively tracked by changes in the maximal cardiac output. In some cases, the severity of heart failure (HF) generally, and the severity of right heart failure in particular, can be effectively tracked by changes in the maximal cardiac output in cases where there may be no change in resting cardiac output.

Accordingly, also provided are analogous methods of monitoring the progression or improvement of HF, particularly right heart failure, in subjects, as well as analogous methods of treating HF, particularly fight heart failure, in subjects to those described herein with respect to COPD. Such methods further comprise obtaining one or more of the PA hemodynamic readings from the subject having HF (e.g., right heart failure) during exercise (e.g., physical exertion such as walking on a treadmill or riding an exercise bike). Methods can further include instructing a subject having HF (e.g., right heart failure) to exercise prior to and/or during the measurement of PA hemodynamic readings. Also provided are methods of assessing the right ventricular function of a subject comprising measuring one or more PA hemodynamic parameters, such as CO, PAP, and TPR, from the subject during exercise, and comparing the parameter to a standard. In some cases, these exercise measurements can be obtained using the ambulatory system described herein.

Systems and Sensors

In the described methods, the PA hemodynamic readings are optionally obtained with a wireless sensor implanted in the subject. Optionally, the implanted sensor is a pressure sensor, which is optionally implanted in the pulmonary artery of the subject. Optionally, the sensor lacks percutaneous connections. Optionally, the sensor is energized from an external source. Optionally, the sensor is a passive sensor energized to return pressure readings by an electromagnetic field.

An effective system and sensor for measurement of PA hemodynamic readings is the CARDIOMEMS (Atlanta, Ga.) heart sensor. As described by U.S. Pat. Nos. 7,699,059 entitled “Implantable Wireless Sensor” and 7,679,355 entitled “Communicating with an Implanted Wireless Sensor,” these sensors are MEMS-based sensors that are implanted in the pulmonary artery, more particularly in the distal pulmonary artery branch and are configured to be energized with RF energy to return high-frequency, high-fidelity dynamic pressure information from a precisely-selected location within a patient's body.

Because of the nature of the sensors described above, the PA pressure waveform is optionally obtained outside of a typical clinical evaluation environment. For example, the CARDIOMEMS sensor can provide ambulatory measurements of hemodynamic parameters, such as PAP, outside of a traditional hospital setting. “Ambulatory measurements” refers to measurements that are made in normal daily-living situations where the patient is not bedridden in a clinical setting. For example, sleeping (e.g., for studies and therapies of sleep apnea), eating and exercise activities at the home or work environments where RHC and other more invasive procedures are largely impractical and/or risky. Ambulatory measurements of PA hemodynamics can be more representative of living conditions of a patient who is suffering from a disease and/or undergoing treatment via a therapeutic regimen. The CARDIOMEMS sensor is non-invasive after implantation, facilitating ambulatory use. For example, in some embodiments, the PA waveform can optionally be obtained while the subject is exercising. Exercising can include any activity of the subject. For example, exercise or exercising can include activities of daily living, prescribed exercise, walking, biking, running or the like. Optionally, the PA pressure waveform is obtained while the subject is asleep.

In some embodiments, the PA hemodynamic readings comprise a high fidelity, real-time PA hemodynamic waveform which can be processed to obtain one or more desired PA hemodynamic parameters. PA hemodynamic waveforms obtained with the CARDIOMEMS sensor are examples of “high fidelity” PA hemodynamic waveforms.

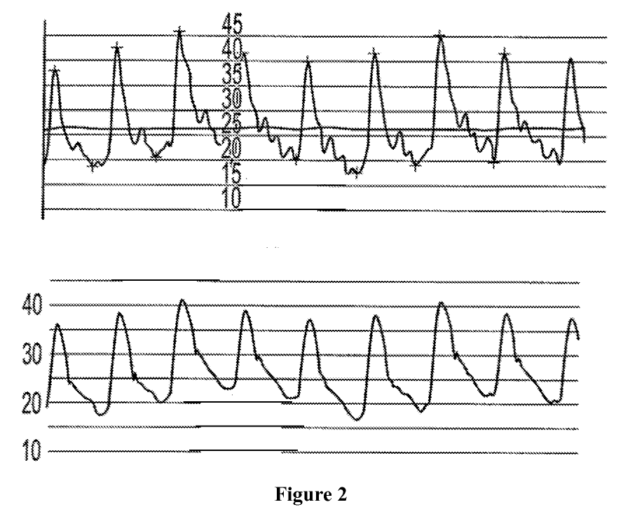

As shown in FIG. 2, PA hemodynamic waveforms measured using other methods, such as right heart catheterization (RHC; top waveform), exhibit undesired whip or overshoot that is believed to exceed systolic and diastolic pressures and which can lead to error and uncertainty. In contrast, the CARDIOMEMS sensor PA waveform (FIG. 2, bottom waveform) exhibits high fidelity through its smooth and undistorted waveform. For example, the dicrotic notch is clear and pronounced compared to the RHC gold standard.

The CARDIOMEMS pressure sensor allows for sampling rates of at least 2,000 samples per second without fluidic artifacts, and can be collected without line occlusion, which is the tendency of the line of a RHC to affect the hemodynamic measurements, and a lack of distortion due to movement that occurs in invasive procedures with long leads or wires extending from the patient. The accuracy of the CARDIOMEMS sensor data are also aided by the addition of resistance effects to the basic Bernoulli model, using Windkessel principles.

The high fidelity waveform is not subject to mechanical artifact with movement, which is commonly observed with conventional invasive blood pressure measurements, using a fluid-filled catheter. Further, the implanted sensor measures absolute pressure and the blood pressure waveform is determined by subtraction of atmospheric pressure, measured by the external measurement system. There is no need for external alignment and leveling of a reference transducer to perform measurements, as is required for conventional invasive blood pressure measurement with a fluid-filled catheter. These features make an implanted wireless sensor especially well-suited for measuring hemodynamic parameters without limitation to activity state or across changes in activity state.

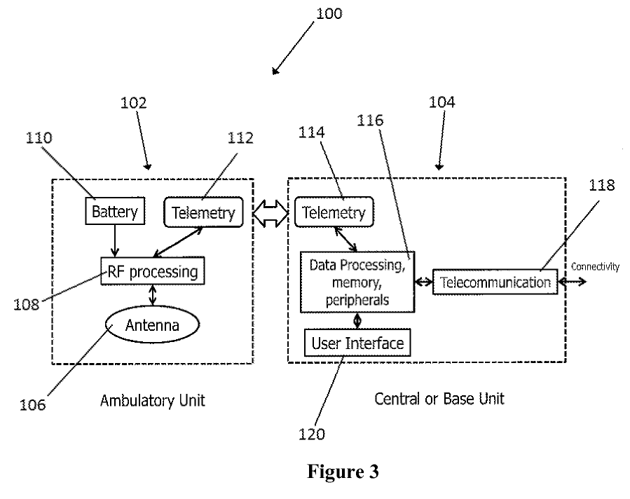

In some embodiments, PA hemodynamic readings are optionally obtained, for example during exercise, using an ambulatory system. Referring now to FIG. 3, an example ambulatory system 100 is shown. The ambulatory system 100 can include an ambulatory unit 102 and a base unit 104. The ambulatory unit 102 and the base unit 104 can be communicatively connected through a communication link. This disclosure contemplates the communication link is any suitable communication link. For example, a communication link may be implemented by any medium that facilitates data exchange between the ambulatory unit 102 and the base unit 104 including, but not limited to, wired, wireless and optical links Optionally, the communication link is a wireless link. For example, the ambulatory unit 102 and the base station 104 can be configured to communicate with each other using a wireless communication protocol such as WiFi, BLUETOOTH or ZIGBEE, for example. It should be understood that other standard or proprietary protocols can optionally be used by the ambulatory unit 102 and the base station 104.

The ambulatory unit 102 includes an antenna 106, an electronics unit 108 (e.g., RF processing unit), a power source 110 (e.g., battery) and a telemetry circuit 112. Optionally, as discussed above, the ambulatory unit 102 can be the external measurement system that is worn by the patient. The antenna 106 is configured to inductively couple energy to a sensor such as the implantable MEMS-based pressure sensor discussed above, for example, and receive return signals from the sensor. The return signals received at the ambulatory unit 102 via the antenna 106 can be processed by the electronics unit 108. For example, the electronics unit 108 can be configured to determine a pressure measurement from the signals as described in U.S. Pat. No. 7,679,355, entitled “Communicating with an Implanted Wireless Sensor.” As discussed below, the processed signal can be transmitted the base unit 104 for storage and/or further processing. In its most basic configuration, the electronics unit 108 can include a processing unit and memory. The memory can optionally be volatile or non-volatile memory or some combination of the two. The processing unit can be configured to perform the arithmetic and logic operations necessary for operation of the ambulatory unit 102. For example, the processing unit can be configured to execute program code encoded in the memory. Additionally, the power source 110 can provide power to the other components of the ambulatory unit such as antenna 106, the electronics unit 108 and the telemetry circuit 112. The telemetry circuit 112 allows the ambulatory unit 102 to communicate with other devices such as the base unit 102, for example, over the communication link. The telemetry circuit 112 can optionally include a transceiver and a microprocessor. Additionally, the transceiver and microprocessor can optionally be the same component. It should be understood that the ambulatory unit 102 discussed above is provided only as an example and that the ambulatory unit 102 can include more or less features than those discussed above.

The base unit 104 includes a telemetry circuit 114, a data processing circuit 116, a network connection 118 and a user interface 120. Optionally, the base unit 104 is a computing device such as desktop computer, laptop computer, tablet device, etc. The telemetry circuit 114 is similar to the telemetry circuit discussed above and is therefore not discuss in further detail below. In its most basic configuration, the data processing unit 116 can include a processing unit and memory. The memory can optionally be volatile or non-volatile memory or some combination of the two. The processing unit can be configured to perform the arithmetic and logic operations necessary for operation of the base unit 104. For example, the processing unit can be configured to execute program code encoded in the memory. Additionally, the network connection 118 allows the base unit 104 to communicate with other devices. For example, the base unit 104 can optionally be connected to a computer network such as a LAN, WAN, MAN, etc. via the network connection 118. Alternatively or additionally, the user interface 120 can include one or more input device (e.g., keyboard, touch screen, mouse, etc.) and/or output devices (e.g., display screen, speakers, printers, etc.). The above components are well known in the art and are therefore not discussed in further detail below. It should be understood that the base unit 104 discussed above is provided only as an example and that the base unit 104 can include more or less features than those discussed above.

Optionally, to allow effective monitoring of patients while active (e.g., during exercise), the ambulatory unit 102 can be designed to be worn on the patient's body. For example, harnessing methods can be used to fix the ambulatory unit 102 properly to the patient. In one example implementation, the electronics unit 108 and battery 110 can be contained within one or more pockets of a garment such as a vest, for example. The electronics unit 108 and the battery 110 can optionally be combined into a single housing or can optionally be housed separately. To provide for the adequate signal integrity, the antenna 106 can be positioned in a fixed position with respect to the sensor (e.g., the implanted MEMS-based sensor). Positioning of the antenna 106 can be flexible to provide for optimal coupling, and therefore, the means of fastening the antenna 106 can provide options for adjusting positioning. These options include movement of antenna 106 physically to another location and/or adjusting/tightening straps to move the relative location of a permanently fixed antenna 106 to a more optimal position. Optionally, the antenna 106 can be sewn in place within a garment such as a vest, or the antenna 106 can be fixed by some means of a temporary fabric fastener such as hook and loop (e.g., VELCRO). Optionally, in the COPD applications, the antenna 106 can be fixed on the patient's back, for instance, over the patient's shoulder blade and can be communicatively connected to the electronics unit 108 with a cable. Alternatively, the antenna 106 can optionally communicate signals received by the antenna 106, for example, over a short range, directly to the base unit 104. In other words, the base unit 104 can be configured to determine a pressure measurement from the signals as discussed above. As discussed above, the ambulatory unit 102 can communicate with the base unit 104 over the communication link, which can be a wireless communication link, for example. The ambulatory unit 102 can optionally be worn by the patient who is located remotely from the base unit 104. The ambulatory unit 102 can communicate data including, but not limited to, data read from the sensors (e.g., the implanted MEMS-based sensors), diagnostic data from the ambulatory unit 102 and barometric pressure. The diagnostic data can include status information such battery status, temperature, etc. This disclosure contemplates that the base unit 104 can communicate with a plurality of ambulatory units at the same time. For example, a plurality of patients, each having an ambulatory unit, can be monitored at the same time. Transmission/reception of data between the base unit 104 and a plurality of ambulatory units can be synchronized to prevent interference. In one implementation, the base unit 104 can control synchronization. For example, each ambulatory unit 102 can be associated with a unique identifier, and the base unit 104 can poll a specific ambulatory unit using its unique identifier to initiate communication. In other words, the base unit 104 can be configured to send a message to an ambulatory unit, and the ambulatory unit can “wake up” and communicate with the base unit 104 upon receipt. Alternatively, the plurality of ambulatory units can control synchronization. For example, each of the ambulatory units can be configured to monitor communications between the other ambulatory units and the base unit 104. The base unit 104 can have a predetermined duty cycle for transmission and reception (e.g., a predetermined transmit-read cycle). The predetermined duty cycle can optionally be 10 microseconds. The base unit 104 can therefore be repeatedly available to receive communications at specified times. It should be understood that the predetermined duty cycle can be more or less than 10 microseconds. The plurality of ambulatory units can synchronize their respective wake periods (e.g., communication periods) into a predetermined order. Alternatively or additionally, the plurality of ambulatory units 102 can agree upon a synchronization order through a networking algorithm. This can optionally be achieved through direct communication from the base unit 104 (e.g., a star network) or through ambulatory unit to ambulatory unit communication in a repeater pattern (e.g., a mesh network).

Optionally, the ambulatory unit 102 can be configured to use one or more duty cycle sequences (e.g., ON/OFF, wake/sleep, etc.) to reduce power requirements. By reducing the power drawn by the ambulatory unit 102, the useful battery life can be extended. In other words, the ambulatory unit 102 can operate for a longer period of time without re-charging its power source 110. The ambulatory unit 102 can be configured to have a “wake” mode in which the ambulatory unit 102 is capable of communicating and/or is communicating with the base unit 104 and a “sleep” mode where its power requirements are reduced. Optionally, in the sleep mode, the ambulatory unit 102 can operate in a lower power state such that power is supplied only to components necessary to “wake-up” the ambulatory unit 102. For example, the telemetry circuit 112 can optionally be configured to send a message to the ambulatory unit 102 to initiate the wake mode. The wake mode-sleep mode cycle can define the duty cycle sequence.

Alternatively or additionally, the ambulatory unit 102 can be configured to implement an intra-sample duty cycle, which can be provided to decrease power requirements of the ambulatory unit 102. The intra-sample duty cycle can be reduced to minimize transmission power of the ambulatory unit 102. The intra-sample duty cycle can optionally be the amount of time the ambulatory unit 102 communicates with/transmits data to the base unit 104 during its wake mode. The intra-sample duty cycle can be optimized for the Q of the sensor (e.g., the implanted MEMS-based sensor) and/or the apparent power of the return signals received from the sensor. For example, the ambulatory unit 102 can optionally optimize the amount of time it communicates with/transmits to the base unit 104. The ambulatory unit 102 can optionally be configured to monitor the amplitude of the return signals received from the sensor and reduce the amount of transmission time (e.g., on-time) while the amplitude of the return signal is below a specified threshold. The specified threshold can correspond to a minimum signal to noise ratio. The ambulatory unit 102 can be configured to reduce transmission time when the signal to noise ratio of the return signal is low because useful data might not be recovered due to the low return signal strength. Additionally, the ambulatory unit 102 can be configured to monitor the amplitude of the return signals received from the sensor and increase the amount of transmission time (e.g., on-time) while the amplitude of the return signal is above a specified threshold.

The base unit 104 can initiate the reading process by accepting the parameter information specific to the patient's sensor, reading parameters (such as length of readings, sample rates, and frequency ranges, etc.) and patient demographic information. The base unit 104 can then communicate the set-up parameters to the ambulatory unit 104, which initiates the reading process. The base unit 104 can also be configured to display the signal strength from the coupled sensor, for example, on the user interface 120. The base unit 104 can optionally be configured to initiate a frequency scan to locate the sensor within a prescribed frequency range if the signal strength is weak. Additionally, the base unit 104 can also be configured to calculate the pressure measured by the sensor given a calibrated pressure sensor during the measurements. The base unit 104 can also be configured to handle data acquisition requests by acquiring several seconds of measured pressure data, which can include systolic, diastolic, pulse, cardiac output and/or mean pressure values for the acquisition period. The base unit 104 can also be configured to store data to memory. Alternatively or additionally, the base unit 104 can also be configured to communicate data such as the wave form and cardiac output, systolic, diastolic, pulse and mean pressure data to a remote computer, for example, a data server of a monitoring system. As discussed above, the base unit 104 network connectable. Optionally, the base unit 104 can be configured to perform the above functions in real-time. Optionally, the base unit 104 can be configured to perform the above functions on a single patient or multiple patients either simultaneously or in succession.

The data from the readings can optionally be accessed and/or analyzed remotely, for instance, through and application accessed through a network (e.g., the Internet) or through an application resident on the base unit 104. The application can allow a user to access data from individual readings or access historical data from individual patients. The application provides the ability to track trends of physiological measurements and associates those trends with events, activity information, other physiological data and medication changes. This data can then be used by a clinician to more effectively treat the patient's disease state.

The ambulatory system can be used to obtain PA hemodynamic readings as part of any of the methods described herein. In certain embodiments, the ambulatory system is used to obtain ambulatory PA hemodynamic readings from a patient (e.g., while the patient is exercising)

### Example 1

### Impact of a Wireless Implanted Pulmonary Artery Pressure Monitoring System in Heart Failure Patients with Comorbid Chronic Obstructive Pulmonary Disease

Chronic obstructive pulmonary disease (COPD) is a common comorbidity for heart failure (HF) patients. The presence of high pulmonary artery pressures are independently associated with COPD and HF exacerbations.

A retrospective analysis was performed to evaluate if PA hemodynamic monitoring and therapy reduced HF hospitalizations (HFH) in HF patients with a medical history of COPD and/or receiving COPD therapies.

The CHAMPION trial enrolled 550 patients with NYHA class III HF who were followed for an average of 15 months. In the treatment group, clinicians used PA hemodynamic data to guide therapy decisions in addition to standard of care versus standard of care alone in the control group.

In the entire CHAMPION cohort, treatment had a 37% reduction in HFH rates (0.46 vs. 0.73, HR 0.63, 95% CI 0.52-0.77, p<0.0001, Anderson-Gill). In the subgroup of 187 patients with comorbid COPD, treatment had a 41% reduction in HFH rates (0.55 vs. 0.96, HR 0.59, 95% CI 0.44-0.81, p=0.0009). Reductions in PAP were analyzed using an area under the curve (AUC) methodology. Overall, treatment had an average AUC reduction of 201.5 mmHg days compared to an increase of 106.5 mmHg days in control (p=0.0299, ANCOVA). In the COPD subgroup, treatment had an average reduction of 353.1 mmHg days compared to a reduction of 57.0 mmHg days in control (p=0.3687).

HF patients with COPD experience high HFH rates but have pronounced benefit from PA hemodynamic monitoring. See Table 1.

TABLE 1
Benefits for HF Patients with COPD From PAP-Guided Medical Treatment

| | | All Patients | | | Patients with COPD | | |
|---|---|---|---|---|---|---|---|
| | | (n = 550; 270 Treatment, 280 Control) | | | (n = 187; 91 Treatment, 96 Control) | | |
| | | Rate | Change | p-value | Rate | Change | p-value |
| HFH | Treatment | 0.46 | −37% | <0.0001 | 0.55 | −41% | 0.0009 |
| | Control | 0.73 | | | 0.96 | | |
| REH | Treatment | 0.07 | −49% | 0.0009 | 0.12 | −62% | 0.0023 |
| | Control | 0.14 | | | 0.31 | | |
| PAP | Treatment | −201.5 | −258.5 | 0.0299 | −353.1 | −300.1 | 0.3687 |
| Modification | Control | 57.0 | | | −57.0 | | |

### Example 2

### Respiratory Event Hospitalizations are Reduced in Heart Failure Patients with Comorbid Chronic Obstructive Pulmonary Disease using a Wireless Implanted Pulmonary Artery Pressure Monitoring System

Respiratory event hospitalizations (REH) from COPD exacerbations, bronchitis, pneumonia, and other respiratory events frequently occur in patients with heart failure (HF). Pulmonary artery pressure (PAP) monitoring and treatment has been shown to reduce heart failure hospitalizations. A retrospective analysis was performed to see if PAP monitoring impacts REH in HF patients who had a medical history of COPD and/or were receiving COPD therapies.

The CHAMPION trial enrolled 550 patients with NYHA class III HF who were followed for an average of 15 months. In all patients PAP data were monitored using a novel, implantable hemodynamic system. In the treatment group, clinicians used PAP data to guide therapy decisions in addition to standard of care versus standard of care alone in the control group. REH rates were higher in the COPD subgroup compared to the entire CHAMPION cohort. In the COPD subgroup (91 treatment vs. 96 control), treatment experienced a 62% reduction in REH rates (0.12 vs. 0.31, HR 0.38, 95% CI 0.21-0.71, p=0.0023, Anderson-Gill). In the CHAMPION cohort (270 treatment vs. 280 control), treatment experienced a 49% reduction in REH rates (0.07 vs. 0.14, HR 0.51, 95% CI 0.44-0.81, p=0.0009). See Table 1.

Treating HF patients with an implantable hemodynamic monitoring system significantly reduced REH in all patients with even greater benefit in COPD patients. Further investigations that analyze the relationship between PAP, COPD, and REH in chronic HF patients and its implication towards new treatment strategies are warranted.

Disclosed are materials, compositions, and components that can be used for, can be used in conjunction with, can be used in preparation for, or are products of the disclosed methods and compositions. These and other materials are disclosed herein, and it is understood that when combinations, subsets, interactions, groups, etc. of these materials are disclosed that while specific reference of each various individual and collective combinations and permutations of these compounds may not be explicitly disclosed, each is specifically contemplated and described herein. For example, if a method is disclosed and discussed and a number of modifications that can be made to a number of molecules including the method are discussed, each and every combination and permutation of the method, and the modifications that are possible are specifically contemplated unless specifically indicated to the contrary. Likewise, any subset or combination of these is also specifically contemplated and disclosed. This concept applies to all aspects of this disclosure including, but not limited to, steps in methods using the disclosed compositions. Thus, if there are a variety of additional steps that can be performed, it is understood that each of these additional steps can be performed with any specific method steps or combination of method steps of the disclosed methods, and that each such combination or subset of combinations is specifically contemplated and should be considered disclosed.

Publications cited herein and the material for which they are cited are hereby specifically incorporated by reference in their entireties.

What is claimed is:

1. A method of predicting the progression or improvement of chronic obstructive pulmonary disease (COPD) in a subject, or predicting an outcome in a subject having COPD, comprising
a. obtaining a pulmonary arterial (PA) hemodynamic reading from the subject, wherein the PA hemodynamic reading comprises a PA hemodynamic waveform;
b. processing the PA hemodynamic waveform to obtain a PA hemodynamic parameter; and
c. comparing the PA hemodynamic parameter obtained from the subject to a standard;
wherein an increase or decrease in the PA hemodynamic parameter as compared to the standard indicates the progression of COPD, the improvement of COPD, the outcome of COPD, or a combination thereof.

2. The method of claim 1, further comprising administering a treatment to the subject, wherein the treatment is selected based on the progression of COPD, the improvement of COPD, the outcome of COPD, or a combination thereof.

3. The method of claim 1, wherein the PA hemodynamic reading includes an ambulatory PA hemodynamic reading.

4. The method of claim 3, wherein the PA hemodynamic reading is obtained wirelessly from an implanted pressure sensor implanted in a pulmonary artery.

5. The method of claim 4, wherein the sensor lacks percutaneous connections.

6. The method of claim 5, wherein the sensor is energized from an external source to return pressure readings by an electromagnetic field.

7. The method of claim 6, wherein the PA hemodynamic reading is taken while the subject is exercising.

8. The method of claim 7, wherein the PA hemodynamic reading is obtained using an ambulatory unit.

9. The method of claim 8, wherein the PA hemodynamic reading comprises a high-fidelity PA hemodynamic waveform.

10. A method of evaluating the progression of heart failure (HF) in a subject comprising obtaining a pulmonary arterial (PA) hemodynamic reading from the subject using an implantable pressure sensor, and comparing the PA hemodynamic reading to a standard,
wherein the PA hemodynamic reading is taken while the subject is exercising, and
wherein an increase or decrease in the PA hemodynamic reading as compared to the standard indicates the progression of HF, the improvement of HF, the outcome of HF, or a combination thereof.

11. The method of claim 10, wherein the heart failure comprises right heart failure.

12. The method of claim 11, wherein the PA hemodynamic reading comprises a high-fidelity PA hemodynamic waveform.

13. The method of claim 12, wherein the PA hemodynamic reading is obtained from the implantable pressure sensor using an ambulatory unit.

14. An ambulatory unit for measuring a physical parameter detected by a wireless sensor, the system comprising:
an antenna providing electromagnetic coupling to the wireless sensor, wherein a signal the physical parameter is received through the antenna;
a processing unit and a memory operably coupled to the processing unit, the memory having computer executable instructions stored thereon that, when executed by the processing unit, cause the processing unit to:
cycle between a communication mode and a low power mode; and
communicate the signal to a remote base unit over a communication link during the communication mode.

15. The ambulatory unit of claim 14, wherein the processing unit is prohibited from communicating the signal to the remote base unit during the low power mode.

16. The ambulatory unit of claim 15, wherein the cycle is determined by the remote base unit.

17. The ambulatory unit of claims 16, further comprising a transceiver for communicating with the remote base unit, wherein the transceiver is configured to initiate the communication mode, wherein the communication mode comprises a transmission window and a non-transmission window.

18. The ambulatory unit of claim 17, wherein a length of the transmission window is related to a characteristic of the signal.

19. The ambulatory unit of claim 18, wherein the characteristic is a signal-to-noise ratio.

20. The ambulatory unit of claim 19, the memory having further computer-executable instructions stored thereon that, when executed by the processing unit, cause the processing unit to decrease the length of the transmission window when the signal-to-noise ratio is below a predetermined threshold.

21. The ambulatory unit of claims 20, wherein the communication link is a wireless communication link.

22. The ambulatory unit of claims 21, the memory having further computer-executable instructions stored thereon that, when executed by the processing unit, cause the processing unit to obtain information related to the signal.

23. The ambulatory unit of claims 22, wherein the wireless sensor comprises an implanted pressure sensor implanted in the pulmonary artery of a patient, and wherein the physical parameter comprises a PA hemodynamic reading.
